(19) United States
(12) Patent Application Publication
(10) Pub. No.: US 2003/0032064 A1
(43) Pub. Date: Feb. 13, 2003

(54) Correction of spectra for subject diversity

(75) Inventors: Babs R. Soller, Northboro, MA (US); Patrick Idwasi, Worcester, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: UMASS/WORCESTER, Worcester, MA (US)

(21) Appl. No.: 10/086,917

(22) Filed: Feb. 28, 2002

Related U.S. Application Data
(60) Provisional application No. 60/272,725, filed on Mar. 1, 2001.
(60) Provisional application No. 60/325,013, filed on Sep. 26, 2001.

(51) Int. Cl. G01N 33/53

(52) U.S. Cl. 435/7.1

(57) ABSTRACT

A non-invasive spectral measurement for a target analyte present in a subject's tissue or blood derives spectral shapes corresponding to one or more human variability factors, such as, skin color, from spectra collected from a diverse calibration group of subjects. Another set of spectra are normalized based on the derived spectral shapes to generate a set of corrected spectra. The corrected spectra are then utilized to generate and/or enhance a calibration model for detecting and/or measuring the target analyte from one or more transderamlly obtained spectra of a subject.

### RELATED APPLICATIONS

The present application claims priority to provisional application entitled “METHOD AND SYSTEM FOR CORRECTION OF SPECTRAL VARIABILITY IN HUMAN SUBJECTS FOR NONINVASIVE MEASUREMENT OF BLOOD OR TISSUE CHEMISTRY,” filed on Mar. 1, 2001, having Serial No. 60/272,725, and provisional application entitled “CORRECTION OF SPECTRA FOR SUBJECT DIVERSITY,” filed on Sep. 26, 2001, having Serial No. 60/325,013, both of which are herein incorporated by reference.

### BACKGROUND OF THE INVENTION

The present invention relates generally to detection and/or measurement of a target analyte in blood or tissue, and more particularly, to non-invasive measurement of such analyte by utilizing spectroscopic techniques.

Much interest has been expressed recently in utilizing spectroscopic techniques, in particular infrared (IR) or near infrared red (NIR) spectroscopy, to non-invasively determine blood or tissue chemistry. Tissue in living subjects, e.g., in human patients, presents an extraordinarily complex medium having many contributing absorbing and scattering materials that affect an interrogating light signal. Factors such as temperature particularly affect IR spectra of various low energy (e.g., hydrogen bond) interaction mechanisms in solution, and drift of components or instrumentation may also result in variations of the sampled spectrum over long and/or separated time intervals.

Some successes in correcting or interpreting spectra obtained from in vivo measurements have been reported by applying statistical methods. These statistical techniques and processing modalities, commonly referred to as chemometrics or multivariate calibration, reduce spectral variability to a linear combination of a small number of component spectra, which can then be used in a calibration equation for determining the identity and/or concentration of a component in an acquired spectrum that is due to the clinical parameter of interest being measured.

Generally, the component spectra are derived empirically by simultaneously collecting a number of spectra over a range of interest, together with the conventionally measured reference values, taken at the time each spectrum is acquired. In applying this technique, one hopes that the most significant spectral variability is due to the clinical parameter of interest, for example, glucose concentration, to allow modeling the principal secondary effects as residual factors. However, when working with tissue, a high degree of uncontrollable variability is necessarily also present, resulting from a variety of structural and chemical constituents of the probed tissue as well as other contributing factors.

In some circumstances, an assumption can be made that the uncontrollable variability is comparable to that present in the set of data originally collected for the development of the calibration equations. In that case, some of the component spectra will model this variability. Thus, for example, it has been shown that when spectrometer drift is minimized, differences among tissue spectra of ten Caucasian subjects, obtained by utilizing transdermal illumination, can be adequately modeled in a calibration equation that allows a spectrographic measurement of hematocrit to be performed through the patients' skin. This measurement apparently succeeds because the confounding factors present in the calibration group are substantially similar to those in the measured subjects.

Unfortunately, one cannot generally validly assume a high degree of homology amongst subjects, or between a group of calibration subjects and an unknown set of future clinical subjects. For example, a calibration equation obtained by modeling a small group of Caucasian subjects, as discussed in the above exemplary case, would not accurately correct spectra for measurement of the same target material in subjects with dark skin. Thus, human variability poses a large confounding influence on the shape and quality of light spectra collected from tissue. As a result, tissue spectroscopy is presently of limited use, and the preponderance of assays and measurements must still be effected by withdrawing and preparing blood, or biopsy of tissue samples, rather than applying a non-invasive light signal to in vivo tissue. To applicants' knowledge, instruments or methodologies have not substantially addressed or alleviated this shortcoming.

Accordingly it is desirable to develop a methodology for calibrating spectrographic measurements for performing accurate NIR and visible spectral measurements of blood and tissue chemistry.

It is also desirable to provide such a calibration technique that is applicable to diverse different clinical parameters of interest.

It is also desirable to develop a spectrographic instrument for accurate detection of a clinical parameter of interest through transdermal spectroscopy.

It is also desirable to develop a spectrographic instrument and methodology that corrects human contributing factors for more accurate detection and analysis of a clinical parameter of interest by utilizing in vivo spectroscopy.

Further, it is desirable to develop a database, or a correction algorithm derived from a database, of human contributing factors and their spectrographic components for use in a general spectrographic instrument.

### SUMMARY OF THE INVENTION

One or more of these and other desirable features are attained in a method of the present invention for performing a non-invasive measurement of a target component present in blood or tissue, such as a native, a diagnostic or a treatment component, wherein the received spectrum is corrected for one or more human tissue contributing factors. The contributing factors may include presence of native pigmentation, body fat, histological constituents or aging effects that influence the spectral signal collected from the tissue.

Methods of the invention proceed by constructing a database of spectral measurements taken from a plurality of subjects. The database includes for each of these subjects both a spectral measurement and an independent assay of of either hematocrit or hemoglobin, as well as a scaled or modeled measurement of a human contributing factor, such as a characteristic of human tissue. In the case of a spectrographic measurement taken through the patient's skin, the contributing factor may be any of a number of factors that affect the transmission, absorbance or scattering properties of the skin. In the case of a spectrographic measurement taken directly at the surface of tissue where there is no intervening dermal layer (e.g., in muscle, organ, or endothelial or mesothelial tissue), the contributing factor may be a condition such as texture, stage (in the case of a disease process or invasive tumor) or other factor affecting the collected spectrum. Some representative human contributing factors include characteristics of skin or tissue such as pigmentation, fat content, or the level of an age- or disease-related condition that affects tissue scattering and absorbance.

In accordance with a further aspect of the present invention, after constructing a database of spectra with a scale indicating the level of the human contributing factor present in the spectra, one then solves for the spectral shape of one or more human contributing factors. In general, to develop a calibration model for a target analyte the spectral absorbance, or reflectance values, are measured for a variety of levels spanning the range of clinical interest, but over a limited range of the human contributing factor or factors. The spectral shapes of the human contributing factors, derived from the database, are then used, either in the construction of an improved calibration model for the target analyte, or with spectra of unrelated clinical subjects assessed with an existing model for the target analyte. The calibration or measurement may be extended to simultaneously address a plurality of human contributing factors by constructing special databases from a number of suitably chosen sub-populations to represent the contributing factors, and the resulting spectral shapes may then be applied to correct a tissue spectrum, both in an initial calibration group or in spectra later acquired from unrelated clinical subjects. The spectral decompositions or transformations from the original database thus correct later spectra for the contributing factor(s) so as to more accurately determine the amount of the target component indicated by the given spectrum. Advantageously, the invention constructs one or more databases to determine the spectral contributions of plural human factors and extends the range of subjects to which the in vivo determinations can be meaningfully applied, e.g., to provide accurate qualitative and/or quantitative assays.

In compiling the database of spectral measurements, one may initially acquire a spectrum from each individual in the database, together with an independent assay for blood hemoglobin or hematocrit. One may also acquire an independent assay for the target component (such as a blood test for glucose) to determine the actual level of the target component, though it is advantageous to keep this level constant in subjects used to construct the database. In addition to acquiring the spectrum, the database includes an estimate of the level of a first human contributing factor (for example, skin pigmentation) present in each individual from whom a spectrum is acquired. Thus, a system to correct for pigmentation can employ CIELAB color values for providing a scale for estimating values of pigmentation. Alternatively, such a scale may be heuristically constructed based on any suitable guidelines; for example, it may be comprised of a number of color-related ethnic categories (e.g., Asian, Caucasian, Mediterranean, Afro-American, Indo-Asian) together with a scale or intensity rating (e.g., a light, medium or dark ranking) within each category. This assigns one of fifteen values of pigmentation to each database sample. Similarly, the scale may be constructed from a quantitative (machine-assessed) measurement of intensity and hue.

In one system, the effects of pigmentation in a multi-ethnic group are corrected by calculating one or more skin color loading vectors using a partial least squares (PLS) regression of a hemoglobin-corrected set of spectra from a multi-ethnic group of subjects with a function of a coordinatized color, such as L, a, b, hue or chroma. Where the CIE L*a*b values are utilized L, a, and b are determined from the reflectance spectra of the skin of each subject. One such system calculates a single loading vector and loading weight vector, or alternatively a plurality of loading vectors and loading weight vectors, by regression on value L_{T}, the log transform of luminance color value L [L_{T}=−log₁₀(L)], producing one or more loading vectors and loading weight vectors, one or more of which may closely resemble the absorbance spectrum of melanin. Other multivariate calibration techniques such as principal component regression or classical least squares can also be applied to the spectra to derive skin color loading and weight vectors. Once derived, the skin color loading and weight vector(s) define transformations that may be applied to normalize an arbitrary set of new spectra, removing or substantially removing dermal artifacts to present corrected spectra that are more readily processed to detect other analytes of interest.

Other human contributing factor databases may be constructed to resolve or correct for other spectral contributions of factors. In the case of a transdermal spectral measurement instrument, these may be relevant skin qualities, such as fat content, or induced scattering due to thickening or the like. For direct tissue spectrometry, a condition such as edema or characteristic cell density may influence the received signal. Each of these factors may be categorized by a correlated objectively measurable clinical criterion. For example the contributing factor of fat content may be estimated by a weight/height ratio, or may be scaled by body surface area, the body mass index, or by a surface area-mass product, or other well correlated measurement parameters. Skin or tissue scattering may be ranked by a measure of age or the like. When the probe is to be applied to tissue directly (rather than through the skin), the contributing factors may relate to the tissue morphology itself. In that case, factors such as surface texture, degree of fibrosis or granularity, or features of a physical pathology or disease manifestation may be identified and quantified. Contributing factors can also be generated to correct for more than one spectral contribution simultaneously. For example, this can be achieved by employing PLS-2 algorithm that allows two variables, e.g., skin pigmentation and body mass index, to be regressed against the orthogonalized data to obtain loading vectors and loading weight vectors.

Following construction of a database in this manner reflecting one or more human contributing factors, their spectral contributions in the collected signal are calibrated by analysis.

A preferred embodiment first removes the hemoglobin contribution to the absorbance spectra by calculating the spectral matrix which is orthogonal to the hematocrit values, using one of a number of known orthogonalization techniques. With the hemoglobin contribution removed, partial least squares (PLS) is used to determine the spectral shapes of each contributing factor.

Once the spectral shapes are derived for the human contributing factors, they can be used with any of a number of methods such as, CLS, PLS, prediction-augmented classical least squares (PACLS), or classical least-squares/partial least-square hybrid algorithms to improve the measurement of target analytes determined from spectra not part of the original calibration data.

Advantageously, when applied to a transdermal spectrographic assay, the present invention quantifies spectral contributions of contributing factors of skin pigmentation, fat content and cell scattering to allow accurate spectrographic determinations of blood or tissue chemistry to be acquired non-invasively through the skin of an arbitrary and unrelated clinical subject, thus extending the range of subjects that may be validly measured with the spectrometer, and enhancing the accuracy of measurement. Once the database of contributing spectra has been constructed, it can be used to minimize the number of spectra required to create accurate calibration models for new target analytes, or to enhance the accuracy of calibration models created without the significant variability in the human factors.

### BRIEF DESCRIPTION OF DRAWINGS

These and other features of the invention will be understood from the description below and claims appended hereto, taken together with the drawings of illustrative embodiments, wherein

FIG. 1 illustrates a spectral probe of the present invention;

FIG. 2 shows a system for spectrographic analysis of the present invention;

FIG. 3 illustrates steps in a method of spectral analysis of according to the teachings of the invention;

FIG. 4 illustrates a set of spectra taken from the forearm of a group of ethnically diverse subjects;

FIG. 5 illustrates three loading vectors obtained by applying the spectral analysis method of the invention to the spectra shown in FIG. 4,

FIG. 6 is a graph illustrating that CIE L value can be accurately measured from a set of reflectance spectra using the derived skin color calibration model,

FIG. 7A illustrates a set of uncorrected reflectance palm spectra of a group of normal and multiethnic subjects,

FIG. 7B illustrates a set of corrected spectra corresponding to those shown in FIG. 7A which have been normalized in accordance with the teachings of the invention,

FIG. 8A illustrates a plurality of spectral shapes corresponding to three factors describing fat content which were derived from body mass index (BMI) by utilizing the teachings of the invention, and

FIG. 8B illustrates the spectral shapes of three factors describing fat content derived from body surface area (BSA) by utilizing the methods of the invention.

### DETAILED DESCRIPTION

The present invention pertains to a spectral analysis system, and associated methods, for providing enhanced identification and/or measurement of constituents, and/or concentrations of constituents, present in a subject's blood or tissue by analyzing spectrographic information collected by applying a probe to the subject.

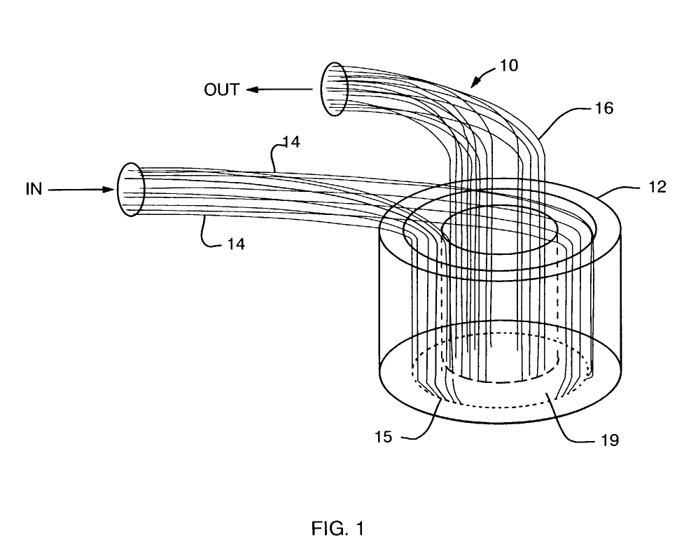

FIG. 1 illustrates a suitable probe 10 useful for the practice of the invention, comprising a plurality of optical fibers arranged to deliver illumination to, and collect return light from, a patient. Probe 10 includes a body having a tissue-contacting probe head 12, in which a first plurality of optical fibers 14 are arranged such that their light-emitting ends are located in a substantially planar ring or annulus 15 extending around the center of the probe head, and are preferably angled inward with a slight radial component. A second plurality of optical fibers 16 have their end faces similarly arranged in small disk-shaped region 19 of the probe head 12 such that when the head 12 is placed against tissue, the fibers 16 receive light that has been directed into the subject by the illumination fibers 14 and has traveled through tissue.

Thus, in use, the probe head 12 is placed against the subject, and the fiber ends are positioned at, or slightly recessed from, the contact surface in a manner to define a tissue interaction path for the collected signal. For use as a transdermal probe that rests against the subject's skin, the outer ring of fibers may be angled inwardly, and positioned such that light penetrates about five millimeters into the underlying muscle, where it is effectively partly absorbed by a constituent of interest, and from which it is reflected back to the detector region 19 and collected by receiving fibers 16. The ends of the light-receiving fibers 16 in the central detection area 19 may be separated from the illumination fibers 14 in the surrounding annulus 15 by a distance of about three millimeters to form an effective tissue probe.

In one embodiment of the probe 10, ninety-one illumination fibers 14, each one hundred micrometers in diameter, are arranged in the illumination ring 15 around the central receiver area 19. Collection is performed by seven optical fibers 16, each one hundred micrometers in diameter, that terminate in the core region 19.

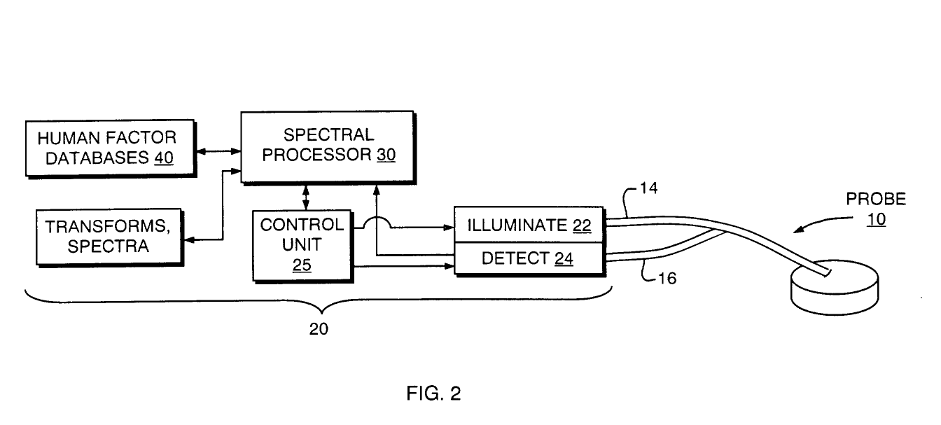

The illustration is not intended to be limiting. The probe head 12 may take various forms effective to deliver and collect light, and to optimize the subdermal absorption component in the collected spectra. It may take a physical form other than the illustrated concentrically-arranged disk-shaped arrays, and may for example, include an adjuster to adjust the angle or positioning of the fibers, or may include a face plate holding the fibers, or a window offset from and protecting the fibers, and may include a baffle or other structure to block direct (non-tissue) light paths between the collecting and receiving fibers, or may include direct illumination or direct detection, rather than fiber interfaces for the source, the detector or both. As further shown in FIG. 2, the probe connects to a spectroscope/processor system that is customized or includes one or more databases and/or spectral analysis modules according to the teachings of the invention that incorporate human contributing factors in the target spectrum, as discussed further below.

The probe connects to a spectrometer system that may generally operate in a known manner to plot or characterize the spectral distribution of the collected light in one of several ways. A broadband tungsten light source may be used to feed the illuminating fibers 14, while the light reflected from underlying tissue in the wavelength band above 400 nanometers is collected by the collection fibers 16 and directed to the detector of a visible and near infrared (NIR) spectrometer. The spectrometer may be a scanning spectrometer, operating with a dispersion element that both separates and directs a single return beam to a photo detector, or may be a non-scanning type incorporating a grating that images the light onto a detector such as a CCD array, to resolve and provide output values for the different wavelengths present in the spectral band. Alternatively, the spectrometer may be an FTIR spectrometer, illuminating with a broad band beam, and spectrally decomposing the collected light analytically by Fourier transformation techniques. Yet another construction is to employ a dispersive element to separate different wavelengths, scan a wavelength-varying component into the illumination fibers 14 so that only a single wavelength illuminates tissue at any given instant, and then simply employ a single light amplitude detector (rather than a CCD or array) to measure the amplitude of the collected signal. In some embodiments, a detector may be placed directly at the probe collection region 19, rather than employing collection fibers 16 and positioning the detector at the distal end of the fibers. In yet another embodiment, the device may employ direct illumination, e.g., one or more light sources with discrete wavelengths (such as light emitting or laser diode) contained in the probe head 12, rather than relying upon a fiber bundle 14 for light delivery to illuminate tissue adjacent the probe head 12.

However, the illustrated system operates with a probe utilizing fiber delivery and fiber collection. This advantageously facilitates use of a remote, cooled photoelectric detector (e.g., a liquid nitrogen cooled CCD or other detector) so that the detection and light collection may be separately optimized for the low signal levels present in the in vivo tissue context. The all-fiber probe embodiment also permits a simple interface with existing spectrometers.

In one implementation, the spectrometer directs a broad-band beam into the fibers 14, collects light in fibers 16 and directs the collected light through a dispersive element which spatially separates the components and directs them at a cooled two-dimensional CCD array to detect the signal intensity. The CCD may be a detector such as a CD12D/512-64 made by Control Development Corp. of South Bend, Ind. The CCD may thus provide outputs for a dispersed 400-1100 nm collected signal that represent spectral intensity of the return signal in half nanometer wavelength steps. Preferably, multiple scans, e.g., ten to fifty scans, are averaged, and the single beam spectra of the reflected light collected from tissue are converted to absorbance units, e.g., log (1/R). Conversion may be effected by taking the ratio to an average value of ten reference spectra collected from a standard, such as the 50% Spectrolan reflectance standard available from Labsphere, Inc. of North Sutton, N.H.

Conceptually, the problem of analyzing the collected light signal to determine the concentration of a given target component is remarkably complex, due in a large measure to the presence of contributing factors in the surrounding tissue (e.g., muscle) or the intervening tissue (e.g., skin). Scattering may occur as a function of the relative size, distribution and optical density of cellular or occult components, and may vary with tissue type, disease states (edema, large-cell processes) and other factors. Similarly, skin pigmentation may affect both scattering and absorption, and may result in its own distinct spectral contributions. Some relatively simple parameters, such as the amount of fat present in tissue, may add a further contribution that also varies between different subjects. The present invention addresses such human contributing factors, and corrects the collected light spectra to enhance accuracy for quantitative spectrometry.

Operation of the system and the component databases and modules of its spectral processor will be better understood from a description of the method of data acquisition and calibration of the present invention. This will be described briefly below with reference to the compilation and construction of spectral databases for correcting for human contributing factors present in light collected transdermally, showing their use in operation of the system as a whole.

Briefly, the technique of the present invention constructs spectral shapes for a number of contributing factors present in the transdermal or tissue environment for which light collection is undertaken. These databases, or component spectra derived from the databases, are then used to process a collected spectral signal for any of a number of different spectrographic assays so as to more accurately quantify the targeted component. Once one or more databases of contributing human factors or confounding spectral influences are constructed, the same processing then allows a general purpose spectral instrument to be readily applied to the detection of other target components without requiring extensive recalibration for each new analyte or for each of the different blood/tissue constituents that are to be targeted.

The elements of an exemplary physical system for practice of the invention are shown in FIG. 2. As shown, a probe 10 as described above is connected to a spectrometer 20, including an illumination component 22 and a detection component 24 coordinated by a control unit 25. The control unit 25 may perform timing, scanning, normalizing, storing and other coordination or signal processing operations appropriate for the type of spectrometer employed, which may be any known spectrometer. The apparatus also includes a microprocessor-based spectral processor 30 operative on the detector output, that processes the received spectral output according to the calibration model, and provides an output, which may be an enhanced assay of the targeted component. The processor 30 communicates with one or more databases 40 that represent or model the effect of one or more confounding human factors, such as tissue scattering, skin pigmentation or the like, discussed herein. These databases may, once constructed, be replaced by a set of stored tables, or be incorporated into calibration equations, constants or transformations derived from the databases, which the processor accesses and applies to modify or process the spectra it receives.

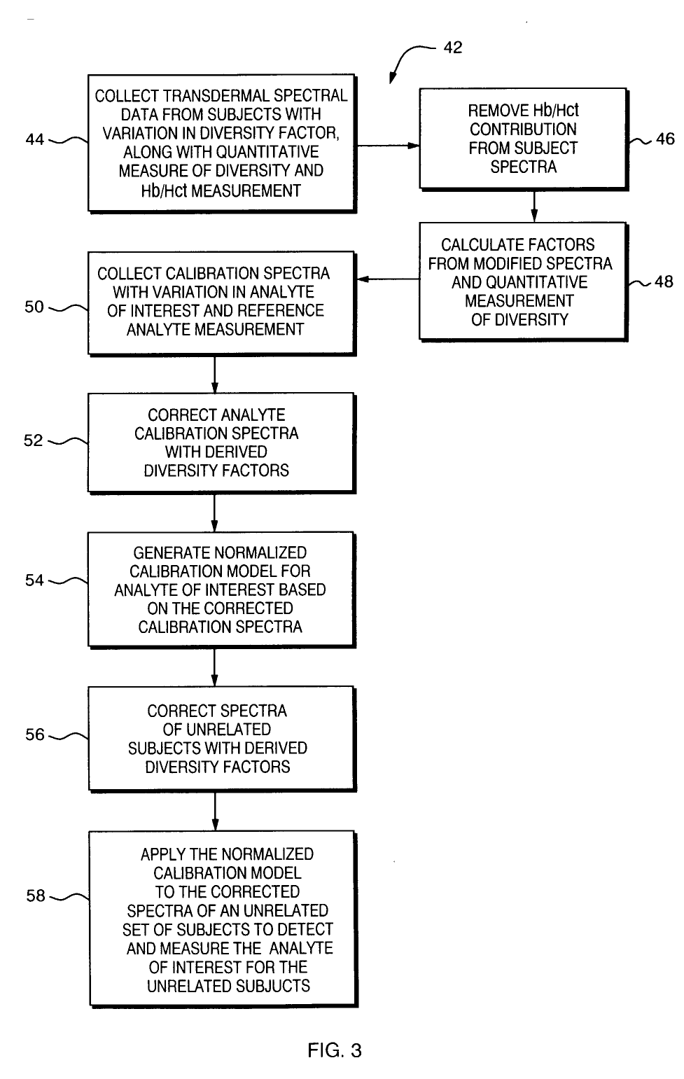

With reference to a flow chart 42 of FIG. 3, in one embodiment of a spectral analysis method according to the invention, in an initial step 44, transdermal spectral data are collected from each subject in a group of subjects with variation in at least one diversity factor, together with quantitative measurements of diversity and hemoglobin and/or hematocrit levels.

In step 46, the contribution of hemoglobin/hematocrit is removed from the subject spectra, for example, in a manner described in Example 1 below, to generate a set of modified (orthogonalized) spectra, i.e., spectra that do not include such contributions. In step 48, one or more contributing factors are calculated based on the modified spectra and quantitative measurement of diversity, as described in detail below.

In the above steps, CIELAB (CIE stands for Commission International d'Eclairage) color values, derived from reflectance spectra of skin, can be utilized to describe a subject's skin color. The data presented in the examples below indicate that CIELAB values provide a valid and statistically significant method for quantitating skin color and ethnic differences. Moreover, the contributing factor of fat concentration may be scaled by relying on a suitable objectively measurable quantity, such as the body surface area (BSA), body mass index (BMI), the weight/height ratio or the like. Skin scattering may be simply represented by age, or an empirical scale based on observed skin texture.

Thus, a database of measurements of selected human factors, and their associated spectral shapes, can be constructed by applying the above methodology to a component development group of subjects, e.g., one hundred or so subjects, for whom these factors, e.g., skin color, scattering and fat content, are scaled. This database can be utilized in different ways to improve the measurement accuracy of a target analyte with models derived from a set of collected spectra.

With continued reference to FIG. 3, in step 50, calibration spectra with variation in an analyte of interest, e.g., glucose, are collected, and a reference analyte measurement is made. The collected calibration spectra are then corrected by utilizing the derived spectral shapes of the diversity factors (step 52).

Subsequently, in step 54, a normalized calibration model, for example, a new partial least squares (PLS) calibration model can be constructed for an analyte of interest. In step 56, the derived spectral shapes (step 48) can be utilized to correct spectra of unrelated subjects, and in step 58, the normalized calibration model can be applied to the corrected spectra of the unrelated set of subjects to detect and/or measure the analyte of interest.

A normalized calibration model based on corrected spectra according to the teachings of the present invention provides superior results for a broad range of subjects because it incorporates corrections for spectral influences of human variability factors.

Alternatively, the derived spectral shapes can be utilized in conjunction with a preexisting calibration model to incorporate subject diversity where none was present during analyte calibration, to generate an enhanced calibration model. The enhanced calibration model can then be employed to detect and/or measure an analyte of interest in one or more unrelated clinical subjects.

The various partial—and classical—least squares (PLS and CLS) calculations for performing one or more of the steps described above may be performed using readily available computational software, such as with the PLSplus/IQ component of the Grams-32 software distributed by Galactic Industries Corp., of Salem, N.H., and suitable Matlab software routines that may be written in the laboratory (Matlab is distributed by MathWorks, Inc., of Natick, Mass.). Comparison of corrected and uncorrected spectral measures and Statistical analysis may be performed with Statistica database management software, available from StatSoft, of Tulsa, Okla.

It should be noted that the special human databases need not be applied to extend a calibration previously constructed from a substantially larger population. An underlying data set for determining a target analyte may be of substantially the same size as one or more of the human contributing factor datasets, or may be considerably smaller. Thus, for example, a spectral database may be formed from a group of ten to twenty homogeneous subjects. In addition to the described techniques for deriving the calibration, similar extensions may be achieved for other common spectral modeling systems, such as principal component regression (PCR) spectral modeling. The calibration extension may also be applied to techniques such as prediction-augmented classical least squares (PACLS), or more recent PACLS/PLS or PACLS/CLS hybrid approaches to produce more accurate results even in the absence of any adequate model the would allow one to quantify the human spectral contributions. The formalism for such processing may follow that described in Haaland D M and Melgaard D K New Prediction-Augmented Classical Least Squares (PA CLS) Methods: Application to Unmodeled Interferents. Appl Spectrosc 54:9, 1303-1312 (2000), and in their article New Classical Least Squares/Partial Least Squares Hybrid Algorithm for Spectral Analyses. Appl Spectrosc 55:1, 1-8 (2001). All of these publications are herein incorporated by reference. Indeed, the calibration of unmodeled human interfering components may proceed by a number of other multivariate techniques, and may, when appropriately validated, be applied to calibration equations augmented by simple clinical estimates that scale the different factors, rather than relying entirely upon fitting with randomized coefficients or similar linear multivariate calibration operations.

Furthermore, the contributing spectral shape of a human diversity component need not be derived by a partial least squares approach using one calibrated target component as described above, but alternatively, may be derived by other means or suitable approximation, for example, as a difference between a limited database calibration spectrum and a human database spectrum, and may then be applied to correct a calibration equation.

Hence, a spectral analysis method according to the present invention allows quick calibration of the in vivo spectral behavior of a target constituent, e.g., an analyte such as glucose, by employing a small set of calibration data based only on varying values of the analyte. The human variability is modeled by preparing and then applying a small number of other special databases that cover a range of one or more human factors that contribute to the subject spectrum. These contributing factor extension databases can then be applied to a calibration model developed for any analyte. The databases may take various forms, such as a set of spectral shapes for each of the human factors being considered. Moreover, the spectrum of the target analyte (such as glucose) may initially be determined in vitro and then corrected and/or used with the human factor contributions to develop a new in vivo calibration model. As described above, a number of different calibration formalisms may be employed.

The step of specifically forming a component database as taught by the present invention is unlike conventional approaches to deriving in vivo spectra in which extension of the calibration would require that an initial calibration be performed with a number of subjects that is so large as to accurately describe or inherently model all patients that will be encountered. Further, unlike the classical approaches that would require that for each new clinical parameter of interest to be measured, similar data be collected from a large and diverse subject population to correct for contributing components, the present invention allows the special database corrections, or the spectra or calibration formulae derived therefrom, to be applied in a straightforward manner to the detection and correction of other target analytes, or to the detection of the specific human factors themselves. The latter property is especially advantageous when a human contributing factor is itself a tissue condition that is indicative of a clinically diagnostic or disease state.

Thus the invention provides a generalized method to correct for spectral variations due to human factors such as skin color, age, or fat content that allows more accurate measurements and further reduces the size of the data set needed to produce calibration equations for the noninvasive measurement of blood and tissue chemistry.

In one embodiment, the methods of the invention are utilized to remove melanin contributions from spectra obtained from retinal pigmented epithelium by utilizing an eye probe/spectrometer, such as that described in co-pending patent application entitled “Ocular spectrometer and probe method for non-invasive spectral measurements,” filed on Feb. 28, 2002 and herein incorporated by reference in its entirety.

The invention and its associated advantages can be further understood by reference to the following examples.

### EXAMPLE 1

Initially, color values were calculated for each of 107 healthy volunteers from a set of forearm reflectance spectra. An analysis of variance (ANOVA), in which the null hypothesis was selected to be that the means corresponding to different groups are the same, was utilized to show that the color values corresponding to people from different groups were statistically different. This analysis was performed in two parts. In the first part, the data was divided according to skin complexion, and in the second part, the data was divided according to ethnicity. Table 1 below presents the result in which the color values in bold letters represent those values that were found to be significant at the 0.05 percent level, i.e., p<0.05. This indicates that the color values are significantly different among the groups into which the subjects are divided. All of the color values, other than CHROMA, were significantly different among different ethnic groups to be suitable for distinguishing subject along ethnic lines. Although in the sample population utilized in this example, CHROMA was found not to be suitable for distinguishing ethnicity. Applicants, however, note that other sample populations may indicate otherwise.

TABLE I

| | | | | | | | | |
|---|---|---|---|---|---|---|---|---|
| Color | SS | df | MS | SS | df | MS | | |
| Value | Effect | Effect | Effect | Error | Error | Error | F | p |

Skin Complexion

| | | | | | | | | |
|---|---|---|---|---|---|---|---|---|
| L | 461.54 | 2 | 230.77 | 1311.20 | 105 | 12.49 | 18.48 | 1.3E−07 |
| A | 103.53 | 2 | 51.76 | 370.25 | 105 | 3.53 | 14.68 | 2.4E−06 |
| B | 91.12 | 2 | 45.56 | 425.97 | 105 | 4.06 | 11.23 | 3.8E−05 |
| CHROMA | 23.67 | 2 | 11.83 | 339.20 | 105 | 3.23 | 3.66 | 2.9E−02 |
| HUE | 1.94 | 2 | 0.97 | 4.00 | 105 | 0.04 | 25.40 | 1.0E−09 |

Ethnic Group

| | | | | | | | | |
|---|---|---|---|---|---|---|---|---|
| L | 558.54 | 5 | 111.71 | 1235.7 | 103 | 12.00 | 9.31 | 2.4E−07 |
| A | 134.62 | 5 | 26.92 | 339.4 | 103 | 3.29 | 8.17 | 1.6E−06 |
| B | 196.61 | 5 | 39.32 | 326.0 | 103 | 3.17 | 12.42 | 1.9E−09 |
| CHROMA | 24.65 | 5 | 4.93 | 339.8 | 103 | 3.30 | 1.49 | 2.0E−01 |
| HUE | 3.02 | 5 | 0.60 | 2.9 | 103 | 0.03 | 21.22 | 1.6E−14 |

Upon determining that color values L, A, B, and hue were useful in distinguishing skin color and ethnicity, these values were utilized together with the same reflectance spectra obtained from the individuals to derive the spectral contribution of skin color. In particular, the procedures described below were utilized to calculate spectral factors ( loading vectors or weights) that describe skin color.

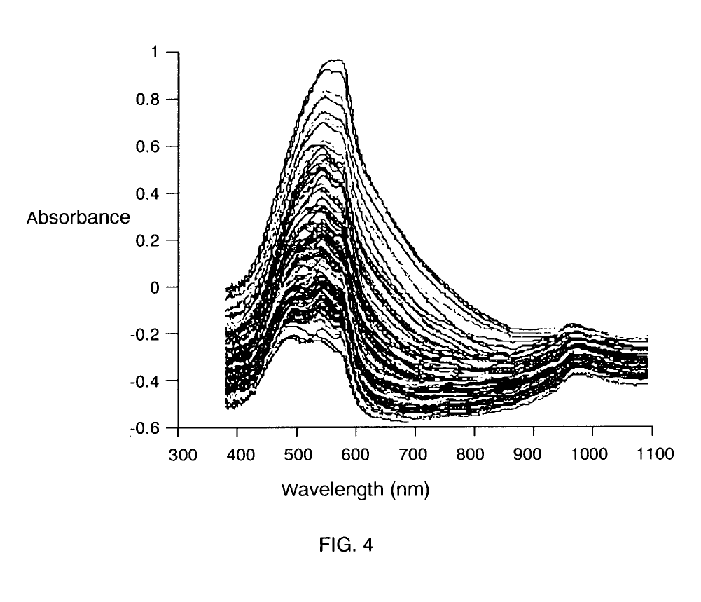

FIG. 4 shows spectra taken from the forearm of the 107 subjects having varying ethnic backgrounds which formed the original data for computing the factors. The collected spectra were first orthogonalized to each subject's hematocrit value to remove the hemoglobin contribution present in the absorbance spectra, by applying an orthogonalization technique to calculate the spectral matrix which is orthogonal to the hematocrit values. This was done using equation (1).

Xₒ=(1−y^{T}(y^{T}y)⁻¹y^{T})X (1)

Here X represents the original spectral matrix (i.e., a matrix of absorbance values at a plurality of wavelengths), y represents hematocrit values for each subject, and X₀ is the spectral matrix resulting from the orthogonalization. ^{T}indicates the transpose of the matrix.

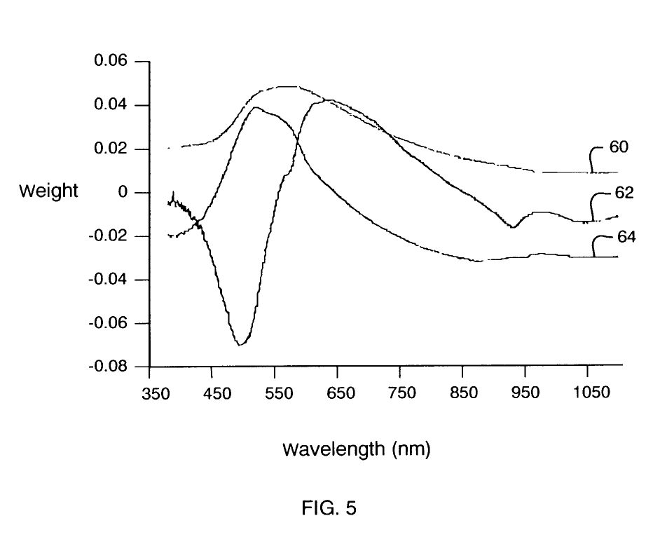

Using the orthogonal spectra Xₒ, applicants next calculated three loading vectors for skin color. Each loading vector p was calculated by carrying out a partial least squares (PLS) regression of the spectra corrected for hemoglobin (X₀) with L_{T}, the log transform of luminance color value L [L_{T}=−log₁₀(L)]. FIG. 5 illustrates the resulting loading vectors in which graph 60 represents a first loading factor, and graphs 62 and 64 represent second and third loading factors, respectively. While a PLS fit was used to calculate the loading vectors, any multivariate calibration technique, such as principal component regression or classical least squares, can also be applied with this data to derive the skin color loading vectors.

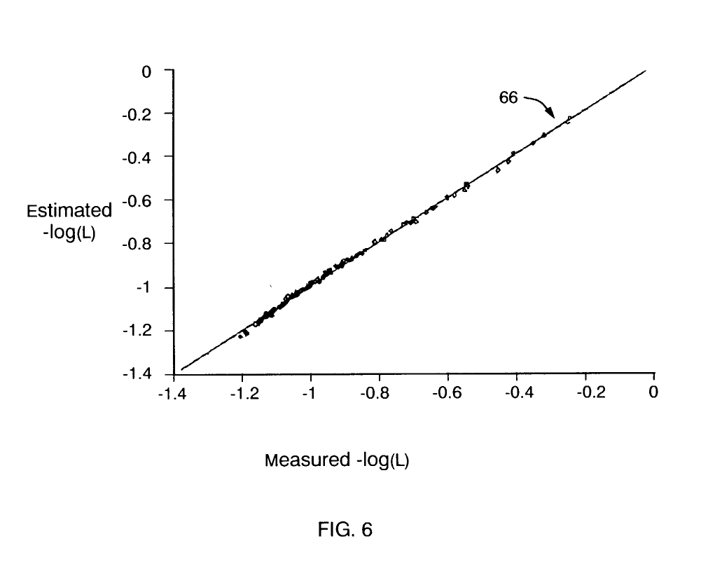

To show that the derived loading factors can be utilized to accurately measure skin color, a regression model was built using a PLS algorithm and full leave-one-out cross validation. The orthogonalized values were used as a response variable (x) and—log(L) values were used as dependent variables (y). FIG. 6 depicts a plot 66 of Estimated—log (L) versus Measured—log(L) which illustrates that the CIE L value can be accurately measured from reflectance spectra by employing the three derived variability factors. In particular, the correlation plot 60 shows a very good agreement between the measured and calculated L values (the R² value for the plot is 0.998).

In accordance with one aspect of applicant's invention, once one or more skin color factors have been derived, one may apply the factor with any multivariate calibration data to derive more accurate calibrations. In the general case (and to correct for other diversity factors, such as inherent system variability), more than one loading vector or weight may be used. If several factors are calculated, they can be represented as a matrix. Thus, given a set of acquired in-vivo spectra, the spectra may be corrected using the loading vector(s) or weight(s). In this example, to correct for skin color in a diverse population, applicants have found three loading vectors provide excellent results, although the use of one loading factor may also be sufficient.

Returning to the example of a skin color correction, the first step of such a procedure is to correct the spectra by calculating how much of each of the skin color loading vectors (p) should be subtracted. This is done as follows. Having taken a new matrix of spectra, the spectra which are to be used for analyte calibration (denoted X_{c}) are multiplied with the loading vector to calculate scores (t) which indicate the amount to be subtracted. That is:

X_{c}p=t_{c} (2)

These scores can also be calculated by utilizing the loading weight vectors (w) rather than the loading vector (p). Then a corrected spectral matrix X_{c}′ is calculated by subtracting out the appropriate amount of the skin color loading vector.

X_{c}′=X_{c}−t_{c}^{T}p (3)

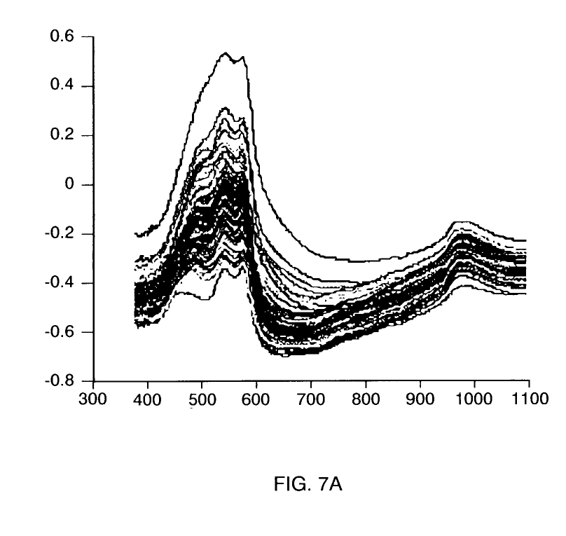

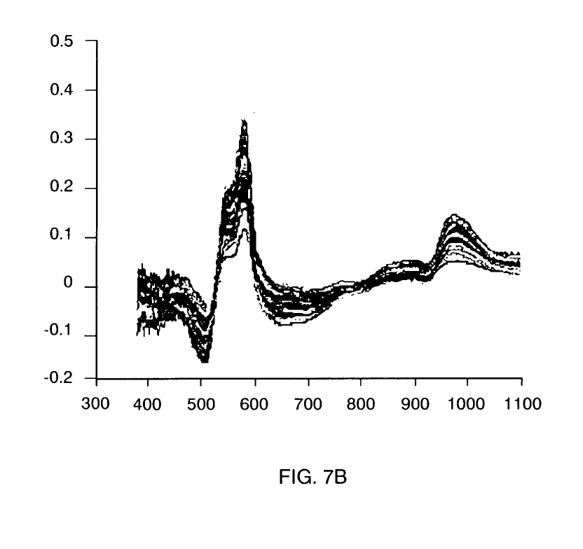

FIGS. 7A and 7B illustrate the effect of correcting a set of transdermally acquired reflectance spectra of 107 multiethnic normal subjects by utilizing the above skin color loading factors. In particular, FIG. 7A illustrates uncorrected palm spectra of these subjects. If the skin color were not a factor, these uncorrected spectra should show only small variations based on hemoglobin concentration because they were all obtained from normal subjects. However, as depicted in FIG. 7A, the uncorrected spectra exhibit a wide variability such that most of the hemoglobin absorption features are obscured by the absorption of melanin. With reference to FIG. 7B, in the corrected spectra corresponding to those shown in FIG. 7A, the hemoglobin features at 549, 581, and 930 nm, as well as the isosbestic point at 800 nm, are clearly visible

The above correction factors were tested for the measurement of hematocrit levels in a completely independent set of patients. In particular, reflectance palm spectra of 18 patients undergoing heart surgery were obtained before and after placement on cardiopulmonary bypasss (CPB), which is known to alter blood hematocrit levels. Blood was drawn to measure the patients' hematocrit levels, and calibration equations were derived to calculate hematocrit levels from uncorrected spectra and from spectra corrected with 1, 2, or 3 factors by employing the procedures described above.

Calibration models were calculated based on subject-on-rotation cross validation. That is, a calibration model was developed from 17 of the 18 patients. In the validation step, spectra from the 18^{th} patient were used in the model to calculate hematocrit. The calculated hematocrit was then compared with hematocrit measured by utilizing a conventional method on blood drawn from the patient. This procedure was performed in turn for each patient. The results for utilizing one, two, or three loading factors are summarized in Table 2 below:

TABLE II
Not on CPB

| Pre-treatment | No. of Spectra | SEP | R² | Bias |
|---|---|---|---|---|
| None | 209 | 1.90 | 0.49 | 0.313 |
| 1 Loading | 211 | 1.93 | 0.52 | 0.248 |
| 2 Loadings | 207 | 1.93 | 0.54 | 0.145 |
| 3 Loadings | 206 | 1.91 | 0.55 | 0.166 |

where SEP denotes standard error of prediction and represents an estimate of the accuracy of the calibration, R2 is indicative of the ability of the model to predict trends in hematocrit, and bias represents an average difference of the result for each patient from perfect prediction for that patient. An inspection of the above Table 2 shows that SEP is less than 2 hematocrit units for all models, and R² improves as additional skin color factors are utilized for correcting the spectra. More importantly, utilizing additional skin color factors for correcting the spectra results in reducing the bias by approximately 50%.

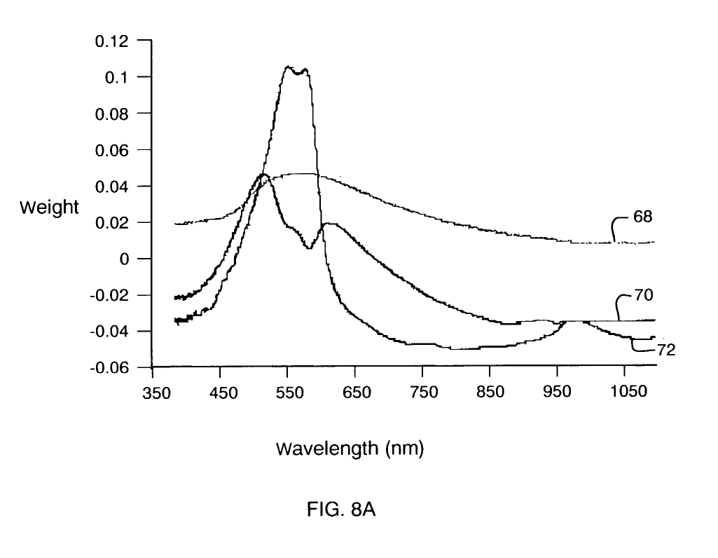

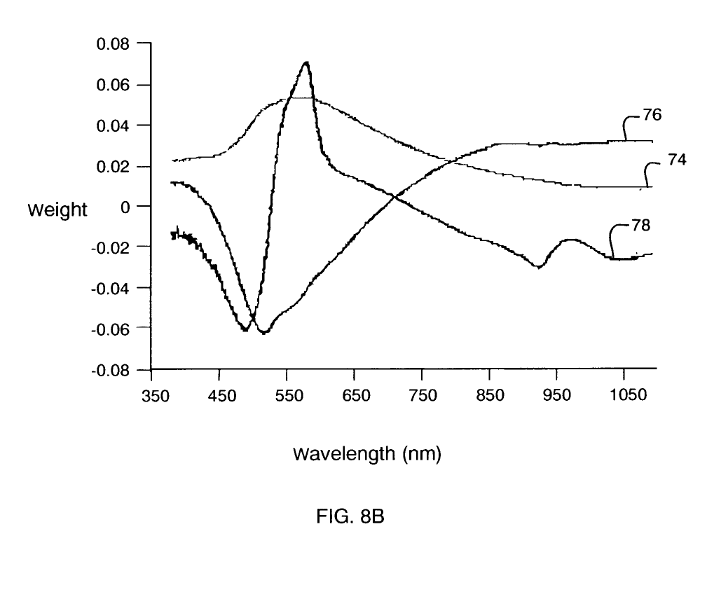

Spectral analysis methods according to the teachings of the invention have general applicability for correcting a wide range of human variability factors. For example, FIGS. 8A and 8B illustrate factors 68, 70, and 72, and factors 74, 76, and 78, respectively, derived by utilizing the method of the invention, that describe fat content. The factors illustrated in FIG. 8A were derived from body mass index (BMI) of 107 multi-ethnic subjects whereas the factors depicted in FIG. 8B were obtained from body surface area (BSA) of the same subjects. It is interesting to note that factor 68 in FIG. 8A is similar to the factor 74 in FIG. 7B, and further these factors are similar to the factor 60 illustrated in FIG. 5 above for skin color correction. Applicants suggest that factors 68 and 74 in FIG. 7A and 7B, respectively, and the factor 60 in FIG. 5 above in fact describe variability in probe placement from one subject to another. This observation is particularly noteworthy because it illustrates that the methods of the invention can be advantageously utilized to correct spectra not only for human factors, such as fat or melanin, but also for variability factors that are inherent in the measurement process.

Thus, by the simple expedient of first defining one or more skin color loading vectors from the hemoglobin-corrected (or hematocrit-corrected) spectra, one is then able to apply the same loading vectors to another set of spectra (e.g., independently-acquired spectra, or new spectra) to normalize the new spectra, reducing the effects of skin color present in the new spectra. Thus, even though the new spectra are acquired transdermally, they are corrected to present a clean and relatively faithful signal for analysis.

In general, applicants expect that by pre-processing the acquired spectral data to correct for skin coloration in this manner, the corrected spectra may be more effectively used in PLS, or any multivariate calibration method to derive a calibration equation for a new analyte. The calibration equation so derived should be more accurate than one derived with uncorrected data, since a large amount of interfering information, due to skin color, is removed. Moreover, once the loading vectors or weights are derived, a corresponding processing software module to correct new transdermally-acquired spectra is readily added to the spectral processing unit of a device.

As a further step, once a normalized calibration equation is derived for the new analyte, a skin color correction may be done on a new spectrum acquired to measure that analyte. Applicants expect, however, that for some analytes of interest, this further step may be unnecessary. To correct the unknown spectrum (denoted Xᵤₖ), the unknown score tᵤₖ may be calculated as in Equation (6). A corrected unknown spectrum (denoted Xᵤₖ′) is defined by Xᵤₖ′=Xᵤₖ−t_{uk.}^{T}p) and is then used in the normalized calibration equation derived in the previous step. Such further correction of the unknown spectrum using the previosly derived skin color loading vector(s) may, in appropriate circumstances, further enhance the accuracy of spectral determinations.

The invention being thus described, variations and modifications will occur to those skilled in the art, and all such variations and modifications are considered to be within the scope of the invention, as described herein and encompassed within the claims appended hereto and equivalents thereof. For example, the methods of the invention can be utilized with spectra obtained at any suitable wavelength. All references cited herein are hereby incorporated by reference in their entirety.

1. A method of performing a non-invasive measurement of a target analyte present in a patient's blood or tissue, the method comprising the steps of:
compiling a database of spectral measurements for a plurality of subjects, the spectral measurements being taken transdermally by utilizing light,
orthogonalizing the spectral measurements to a known chromophore measured in each of the subjects, thereby producing a set of orthogonalized spectral measurements, and
deriving spectral shapes corresponding to one or more variability factors based on said orthogonalized spectral measurements.

2. The method of claim 1, further comprising the step of collecting calibration spectra with variation in the target analyte.

3. The method of claim 2, further comprising the step of utilizing the derived spectral shapes to correct the collected calibration spectra.

4. The method of claim 2, further comprising the step of generating a calibration model based on said corrected calibration spectra.

5. The method of claim 1, wherein the orthogonal spectral measurements are coordinatized, and the step of deriving spectral shapes includes the step of applying a multivariate calibration technique to a coordinate.

6. The method of claim 4, wherein said variability factor is selected to represent variability in any of skin color, fat content derived from body mass index (BMI), fat content derived from body surface area (BSA), age or a disease condition among the subjects.

7. The method of claim 5, wherein the coordinate includes one or more of L, a, b, hue and chroma and a function thereof.

8. The method of claim 4, wherein the coordinate is luminance in a CIE L*a*b spectral representation.

9. The method of claim 7, wherein the coordinate is a coordinate in a CIE L*a*b spectral representation or a quantity calculated from one or more said coordinates.

10. The method of claim 1, wherein said at least one spectral shape is applied to normalize a set of spectral data forming a calibration model for a target analyte to thereby enhance accuracy of in vivo spectral detection of the target analyte.

11. The method of claim 1, wherein said variability factor is selected to represent variability inherent in measurement process.

12. The method of claim 4, further comprising the steps of:
collecting one or more new transdermally obtained spectra from a group of subjects unrelated to the subjects employed for compiling the database,
correcting the new spectra based on the derived spectral shapes, and
applying the calibration model to the new corrected spectra to measure the target analyte.

13. A method of performing a non-invasive measurement of a target analyte present in a subject's blood or tissue, comprising:
compiling a database of transdermally collected spectral measurements for a plurality of subjects,
deriving spectral shapes corresponding to one or more human contributing factors from said collected spectra, and
normalizing the collected spectra based on the derived spectral shapes to generate a set of corrected spectra.

14. The method of claim 12, wherein said variability factor represents variability in any of skin color, fat content derived from body mass index (BMI), fat content derived from body surface area (BSA), age, and disease condition.

15. The method of claim 12, further comprising the step of enhancing a calibration model based on said corrected spectra for measuring the target analyte.

16. The method of claim 12, wherein said human contributing factors are selected to be any of skin color, fat content, or cell scattering.

17. A method of performing a non-invasive measurement of a target analyte present in a subject's blood or tissue, comprising:
compiling a database of transdermally obtained spectra for a plurality of subjects,
deriving spectral shapes corresponding to one or more human contributing factors from said transdermally obtained spectra,
normalizing the transdermally obtained spectra based on the derived spectral shapes to generate a set of corrected spectra, and
utilizing the corrected spectra to augment a calibration model.

18. A spectrometer for in vivo analysis of a target analyte present in a subject's blood or tissue, comprising
a light source and a light collector for, respectively, illuminating and collecting light from the subject's tissue and/or blood to provide a transdermal tissue spectrum, and
a processor operative on the spectrum to measure the target analyte,
wherein said processor operates with a calibration model based on a set of spectra obtained from a group of subjects having variable analyte concentrations and normalized by one or more spectral shapes indicative of one or more human variability factors.

19. The spectrometer of claim 18, further comprising a wavelength dispersing element coupled to the light collector for obtaining the tissue spectrum.

20. The spectrometer of claim 18, wherein the human variability factors can be any of skin color, fat content, age or disease condition.

21. A spectrometer for in vivo analysis of a target analyte, said spectrometer including a light source and light collector for illuminating and collecting light from tissue, and a processor operative on the collected light, wherein said processor operates to correct collected spectra with a transformation determined from spectral measurements taken in a selected population group, wherein the selected population group is a group selected to model spectral contribution of a human contributing factor, thereby enhancing detection of the target analyte in the presence of said human contributing factor.
